(19) United States
(12) Patent Application Publication
(10) Pub. No.: US 2007/0208220 A1
(43) Pub. Date: Sep. 6, 2007

(54) Endoscopic delivery apparatus having a catheter with radial grooves

(75) Inventor: Matthew P. Carter, Dobson, NC (US)

Correspondence Address: BRINKS HOFER GILSON & LIONE/CHICAGO/COOK, PO BOX 10395, CHICAGO, IL, 60610, US

(73) Assignee: WILSON-COOK MEDICAL INC., WINSTON-SALEM, NC (US)

(21) Appl. No.: 11/713,939

(22) Filed: Mar. 5, 2007

Related U.S. Application Data
(60) Provisional application No. 60/779,036, filed on Mar. 3, 2006.

(51) Int. Cl. A61B 1/00

(52) U.S. Cl. 600/107; 600/106; 604/165.01

(57) ABSTRACT

An apparatus cooperable with a grasping slot of an elevator for an endoscope is disclosed. The apparatus comprises an outer catheter including an inner lumen and an outer surface having a radial groove formed thereabout. The radial groove is cooperable with the grasping slot to inhibit longitudinal movement of the catheter with respect to the elevator. The apparatus further comprises an elongate member disposed through the inner lumen of the catheter.

### CROSS-REFERENCE TO RELATED APPLICATIONS

This application claims the benefit of U.S. Provisional Application Ser. No. 60/779,036, filed on Mar. 3, 2006, entitled “ENDOSCOPIC DELIVERY APPARATUS HAVING A CATHETER WITH RADIAL GROOVES,” the entire contents of which are incorporated herein by reference.

### FIELD OF THE INVENTION

The present invention relates to medical devices, and more particularly, to endoscopic delivery apparatus.

### BACKGROUND OF THE INVENTION

Endoscopic devices have been commonly used for various procedures, typically in the abdominal area. Endoscopy is the examination and inspection of the interior of body organs, joints or cavities through an endoscope. Endoscopy allows physicians to peer through the body's passageways. An endoscopic procedure may be used to diagnose various conditions by close examination of internal organ and body structures and may also guide therapy and repair, such as the removal of torn cartilage from the bearing surfaces of a joint. A biopsy, a procedure involving tissue sampling for pathologic testing, may also be performed under endoscopic guidance. For example, endoscopic procedures include the following known procedures: gastroscopy, sigmoidoscopy and colonoscopy, esophago gastro duodenoscopy (EGD), endoscopic retrograde cholangiopancreatography (ERCP), and bronchoscopy.

The use of endoscopic treatments has recently increased for some diseases occurring in the gastrointestinal or pancreatobiliary duct systems. Endoscope systems are used frequently for diagnostic procedures, including contrast imaging of biliary or pancreatic ducts. Endoscopes are also used in procedures for retrieving gallstones that exist in the common bile duct and elsewhere.

Typically, these treatments are performed in the pancreatic duct, bile duct, and the hepatic duct by positioning the distal end of an endoscope in the vicinity of the duodenal papilla. Once the endoscope is in place, a wire guide is delivered to the target anatomy via the working channel of the endoscope. In order to guide the wire guide (or other medical instruments) out of the working channel of the endoscope, a rigid elevator is typically used to orient or deflect the distal end of the wire guide. When the distal end of the wire guide is properly oriented, the wire guide is inserted into the target anatomy.

At this point in the procedure, a catheter or similar treatment instrument can be passed over the wire guide either in a conventional over-the-wire style or in a rapid exchange style to the target anatomy. In order to limit movement of the wire guide relative to the target anatomy, the distal or proximal ends of the guide wire can be locked relative to the endoscope.

Many current endoscopic systems include endoscopes having an elevator used to orient the wire guide and to lock the distal end of the wire guide. In many of such endoscopes, the elevator includes a v-shaped groove. The v-shaped groove is typically used to guide the wire guide to a central position relative to the endoscope. The elevator having a v-shaped groove is further used to lock the distal end of the guide wire.

Thus, there is a need to hold or maintain a delivery device, such as a catheter, within an endoscope so that another device such as a stent may be delivered to a target location in a patient anatomy.

### BRIEF SUMMARY OF THE INVENTION

The present invention generally provides an endoscopic assembly and an endoscopic delivery apparatus that solve the challenges mentioned above. The present invention provides a way of holding or maintaining a delivery apparatus in a relatively fixed position so that a medical device, such as a stent, may be delivered to a target location in a patient anatomy. Embodiments of the present invention provide for a way to rest or seat a delivery apparatus on an elevator of an endoscope during deployment of a medical device without compromising time effectiveness.

In one embodiment, the present invention provides an apparatus cooperable with a grasping slot of an elevator for an endoscope. The apparatus comprises an outer catheter including an inner lumen and an outer surface having a radial groove formed thereabout. The radial groove is cooperable with the grasping slot to inhibit longitudinal movement of the catheter with respect to the elevator. The apparatus further comprises an elongate member disposed through the inner lumen of the catheter.

In another embodiment, the present invention provides an endoscopic system. The system comprises an insertion tube extending to a distal tip and including an elevator movably attached thereto. The elevator has an inner side formed thereon defining a grasping slot. The system further comprises an outer catheter including an inner lumen and an outer surface having a radial groove formed thereabout. The radial groove is cooperable with the grasping slot to inhibit longitudinal movement of the catheter with respect to the elevator. The system further comprises an elongate member disposed through the inner lumen of the catheter.

In another example, the present invention provides a method of engaging an apparatus cooperable with an elevator of an endoscope. The method comprises inserting a catheter in a patient anatomy. The catheter includes an inner lumen and an outer surface having a radial groove formed thereabout. The radial groove is cooperable with the grasping slot. The method further comprises positioning the outer catheter within a target location in the patient anatomy and engaging the radial groove with the elevator to inhibit longitudinal movement of the catheter with respect to the elevator.

Further objects, features, and advantages of the present invention will become apparent from consideration of the following description and the appended claims when taken in connection with the accompanying drawings.

### BRIEF DESCRIPTION OF THE DRAWINGS

FIG. 1a is a perspective view of an endoscopic system comprising an endoscope in accordance with one embodiment of the present invention;

FIG. 1b is a perspective view of the endoscope depicted in FIG. 1A;

FIG. 1c is an elevated view of a distal tip of the endoscope in accordance with one embodiment of the present invention;

FIG. 2 is an enlarged view of the distal tip of the endoscope in accordance with one embodiment of the present invention;

FIG. 3 is a cross-sectional view of the distal tip of the endoscope insertion portion of the endoscope taken along line 3-3;

FIG. 4 is a side view of an elevator in accordance with one embodiment of the present invention;

FIG. 5 is a side view of a delivery apparatus in accordance with one embodiment of the present invention;

FIG. 6 is a schematic view of the delivery apparatus in accordance with one embodiment of the present invention; and

FIG. 7 is a perspective view of the delivery apparatus cooperable with an elevator of an endoscope.

### DETAILED DESCRIPTION OF THE INVENTION

The present invention generally provides an endoscopic delivery apparatus and an endoscopic assembly having features that allow the delivery apparatus to be held or maintained in a particular longitudinal position so that a medical device (such as a stent or coil) may be delivered to a target location in a patient anatomy. Embodiments of the present invention allow a practioner to focus on movement of a stent or other device to be deployed at the target location without relative concern of variable movement of the delivery apparatus. In one embodiment, the endoscopic delivery apparatus comprises an outer catheter having a plurality of grooves formed thereon. The grooves cooperate with an elevator of the endoscope so that the outer catheter may be seated or held in a longitudinal position relative to the endoscope, thereby allowing a medical device to be deployed to a target location in a patient anatomy.

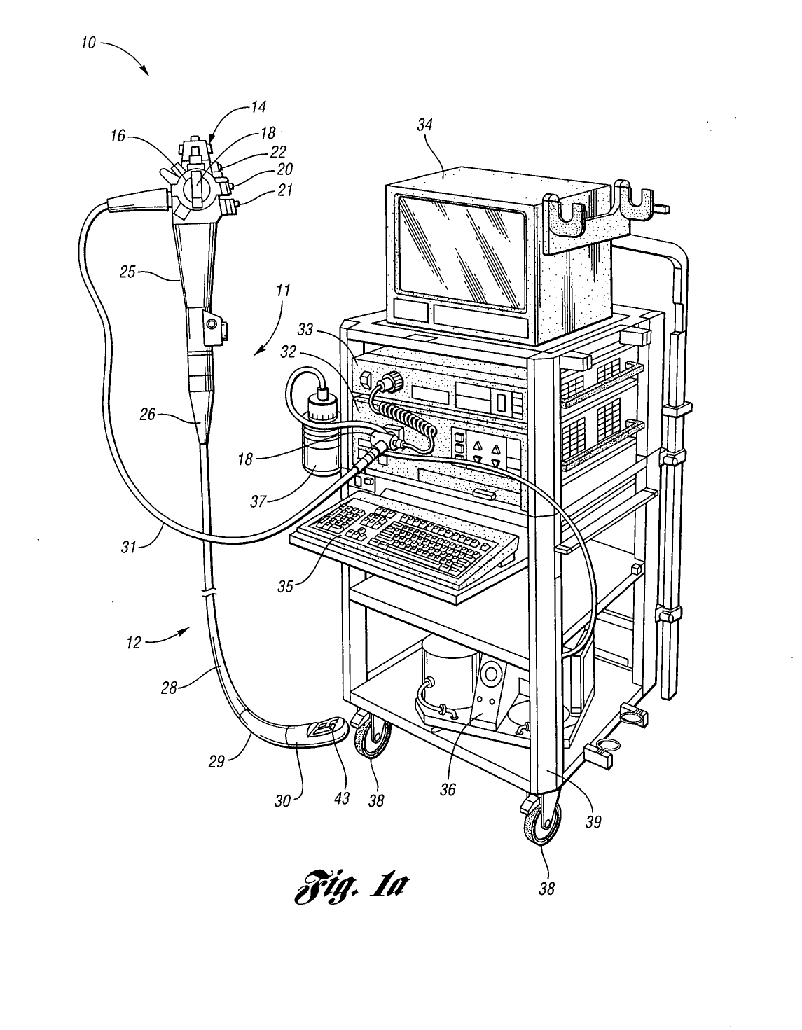

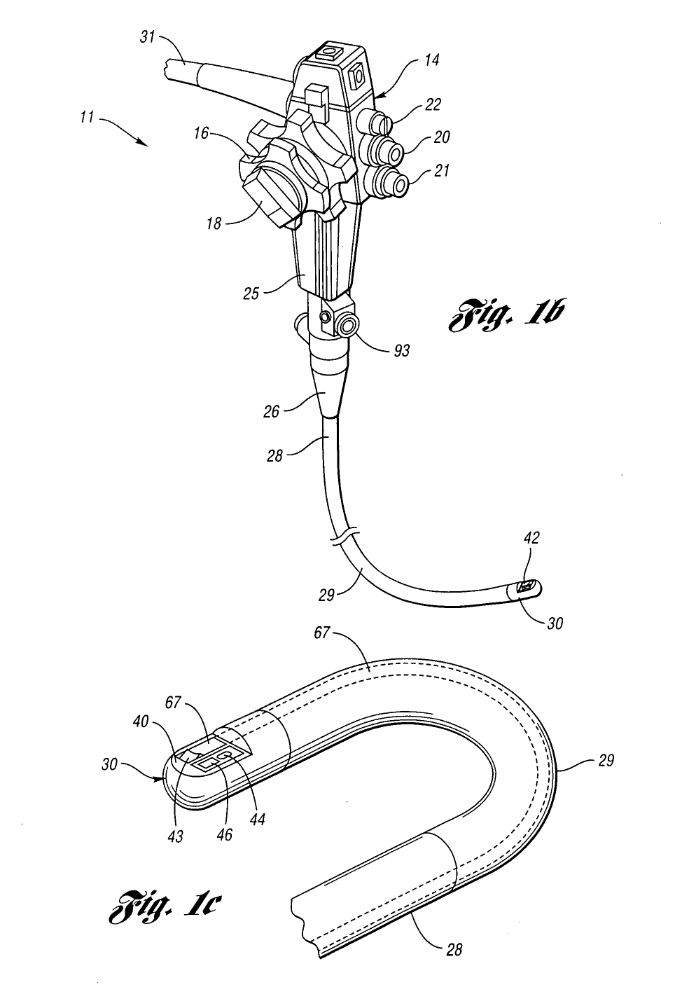

FIGS. 1-3 illustrate an endoscopic system comprising an endoscope having an elevator with a distal tip. In one example, this system represents a modification to the Olympus V-Scope™. Additional details relating to the endoscopic system discussed herein are described in U.S. Pat. No. 6,827,683, entitled “ENDOSCOPE SYSTEM AND MEDICAL TREATMENT METHOD” issued Dec. 7, 2004 to Takashi Otawara, which is incorporated herein by reference in its entirety.

FIG. 1a illustrates an endoscopic system 10 comprising an endoscope 11 in accordance with one embodiment of the present invention. In this embodiment, the endoscope 11 comprises an insertion tube 12 to be inserted into a body cavity for various endoscopic procedures including gastroscopy, sigmoidoscopy and colonoscopy, esophago gastro duodenoscopy (EGD), endoscopic retrograde cholangiopancreatography (ERCP), and bronchoscopy. The insertion tube 12 has an accessory port through which endoscopic units may be disposed. In one embodiment, endoscopic units disposed in one of the accessory ports may include one embodiment of an improved elevator having a distal tip.

As shown in FIGS. 1a and 1b, the endoscope 11 further includes a control system 14 that is in mechanical and fluid communication with the insertion tube 12. The control system 14 is configured to control the insertion tube 12 and endoscopic parts disposed therein. As shown, the control system 14 includes first and second control knobs 16, 18. The control knobs 16, 18 are configured to be in mechanical communication with the insertion tube 12. The control knobs 16, 18 allow the physician to control and guide, by known means, the insertion tube 12 through vessels and cavities of a patient. The control system 14 further includes valve switches (e.g., suction valve 20, air/water valve 21, camera valve 22), each of which are in communication with one of the channel ports 93 of the insertion tube 12. For example, the suction valve switch 20, when activated, allows a vacuum from a suction source through a suction channel port for suctioning unwanted plaque and debris from the patient. Insertion of the insertion tube 12 may be rectally or orally depending on the endoscopic procedure. In one example, the distal end of the insertion tube 12 is inserted, rectally or orally, to a predetermined endoscopic location within a patient. The endoscope in combination with the elevator having the distal tip reduce the risk of tearing or scraping of the wire guide.

In this embodiment, the insertion tube 12 comprises an operating portion 25 connected to the control system 14 and extending to an insertion protecting member 26. A control system 14 is connected to the operating portion 25 and is configured to control the insertion tube 12. In this embodiment, the insertion tube 12 is composed of components that include a flexible tube 28, a flexure 29 connected to the flexible tube 28, and an endoscope tip 30 connect to the flexure 29. A universal cord 31, on one end, is connected and in communication with the control system 14. On the other end, the cord 31 has a connector 18 attached thereto. The connector 18 is in communication to a light guide tube and electrical contact, and is connected to a light source apparatus 32 and an image processing apparatus 33 (external devices). These external devices may include a monitor 34, an input keyboard 35, a suction pump apparatus 36, an irrigation bottle 37, and other suitable apparatus that are installed on a rack 39 equipped with rollers 38.

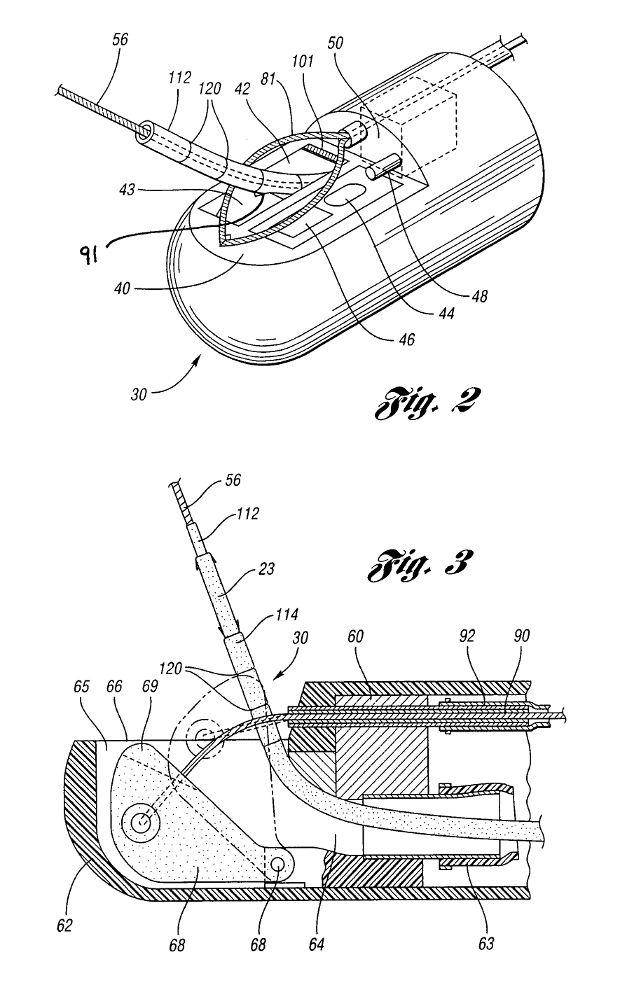

As shown in FIGS. 1c and 2, a concave, depressed cutout 40 is formed on the outer circumferential surface of the tip 30. In this embodiment, a channel opening 42 is formed on one side of the cutout 40, and an objective lens 44 and a light source 46 are disposed on another side of the cutout 40 for imaging. Both the objective lens 44 and the light source 46 are positioned adjacent to the channel opening 42. The tip 30 further comprises a nozzle 48 extending from a back wall surface 50 of the cutout 40. The nozzle 48 allows a stream of water, air, or the like to be sprayed towards the outer surface of the objective lens 44 to clean the lens surface.

FIG. 3 further illustrates the elevator 43 comprising a grasping slot 91 in accordance with one embodiment of the present invention. The grasping slot may take on any suitable shape or form for grasping of a medical device. In this embodiment, the grasping slot 91 is narrowly formed by inner sides that define the grasping slot 91 formed through the elevator 43. Preferably, the grasping slot 91 is centrally formed through the elevator 43 for receiving a medical device (e.g., an inner catheter or wire guide) and grasping the device during operation of the endoscope.

As depicted in FIG. 2, system 10 further includes an outer catheter 112 disposed through the insertion tube 12 and a wire guide 56 disposed through the catheter 112. The elevator 43 is configured to receive the catheter 112 and/or wire guide 56 for elevating the catheter 112 or wire guide 56. As will be described in greater detail below, the delivery apparatus of the present invention has radial notches or grooves formed on the outer surface of the catheter to hold or maintain the catheter in a relatively fixed longitudinal position while other components may be moved relative thereto. For example, as the catheter is held in the grasping slot of the elevator, the wire guide or inner catheter may be withdrawn to deploy a stent or a coil to a target location in the patient anatomy.

The elevator 43 is pivotally attached to the tip 30 and is configured to receive the medical instrument (e.g., catheter or wire guide) for elevating the medical instrument. As shown in FIGS. 2-4, the distal tip houses the elevator 43 in channel opening 42. The elevator 43 is used to orient medical instruments such as a catheter. As discussed in greater detail below, this is accomplished by engaging the medical instrument and pivoting away from the distal tip thereby laterally moving the distal end of the medical instrument away from the distal tip. The elevator 43 thus secures the distal end of the medical instrument relative to the endoscope. That is, as the medical instrument is received in slot 91 of the elevator 43, the medical instrument laterally moves relative to the tip 30 when the elevator 43 pivots therefrom.

FIG. 3 illustrates that the endoscope tip 30 includes a cuff 60 as the main body of the tip 30, and a sleeve or cover 62 that covers the perimeter of the cuff 60. As shown, the cover 62 is formed using a nonconductive member such as any suitable polymeric material, e.g., polytetrafluoroethylene, polyethylene, polypropylene, perfluoroelastomer, fluoroelastomer, nitrile, neoprene, polyurethane, silicone, styrene-butadiene, rubber, and polyisobutylene. In this embodiment, the cover 62 is attached to the cuff 60 by any suitable means, e.g., by adhesive bonding. The cuff 60 is disposed adjacent the working channel 63, which acts as a passageway for the insertion of the medical instrument, e.g., wire guide or catheter. In this embodiment, a channel 67 (FIG. 1c) is formed through the tip 30 such that the tip opening of the treatment instrument is able to be disposed through channel opening 42.

FIG. 3 further illustrates an elevator wire 90 connected to the elevator 43. In this embodiment, the elevator wire 90 is located at the operating portion 25 and extends through a guide tube 92 and a guide pipe 93 connected to the guide tube 92. The elevator wire 90 is in mechanical communication with the control system 14 so that manipulations at the control system 14 result in movement of the elevator wire 90 relative to the endoscope. FIG. 3 depicts (in phantom) movement of the elevator 43 when the elevator wire 90 is actuated at the control system 14, moving the position of the elevator 43 about the elevator turning support 68 as the elevator wire 90 is retracted or pulled.

FIG. 5 illustrates an outer catheter 112 in accordance with one embodiment of the present invention. As shown, the outer catheter 112 includes an outer surface 114 having axial notches or grooves 120 formed thereon. In this embodiment, each of the grooves 120 is formed about a portion of the outer surface 114 of the outer catheter 112. The formation of the grooves 120 allows the outer catheter 112 to be held or maintained within the grasping slot of the elevator, thereby allowing other components of the delivery apparatus to be moved relative thereto. For example, as shown, the wire guide 56 may be longitudinally moved to allow deployment of a stent 123 at the target location in the patient anatomy. Other uses may include holding the catheter within the grasping slot of the elevator such that an inner catheter may be advanced therethrough.

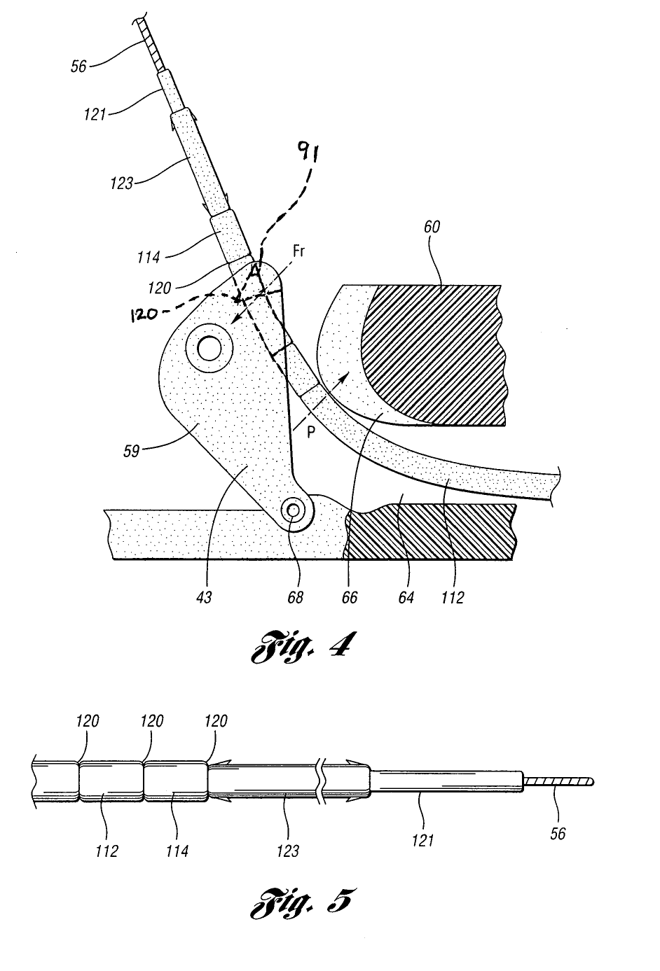

In this embodiment, the elevator 43 is moved about the elevator turning support 68 by manipulating or actuating the control system 14 to pull or retract the elevator wire 90. As the grooves 120 engage and cooperate with the elevator, the outer catheter 112 moves in the direction of the arrow P and pushes the elevator 43 against the cuff 60 (see FIG. 4). Because the outer catheter 112 is relatively axially stiff, it tends to remain straight when pushed against the cuff 60, creating a reactive force in the direction of the arrow Fr in FIG. 4. By means of this reactive force, the catheter 112 is pressed against the slot 91. Moreover, as the elevator 43 and the cuff 60 press against one another, the grooves 120 of the catheter 112 maintain the catheter engaged with the elevator.

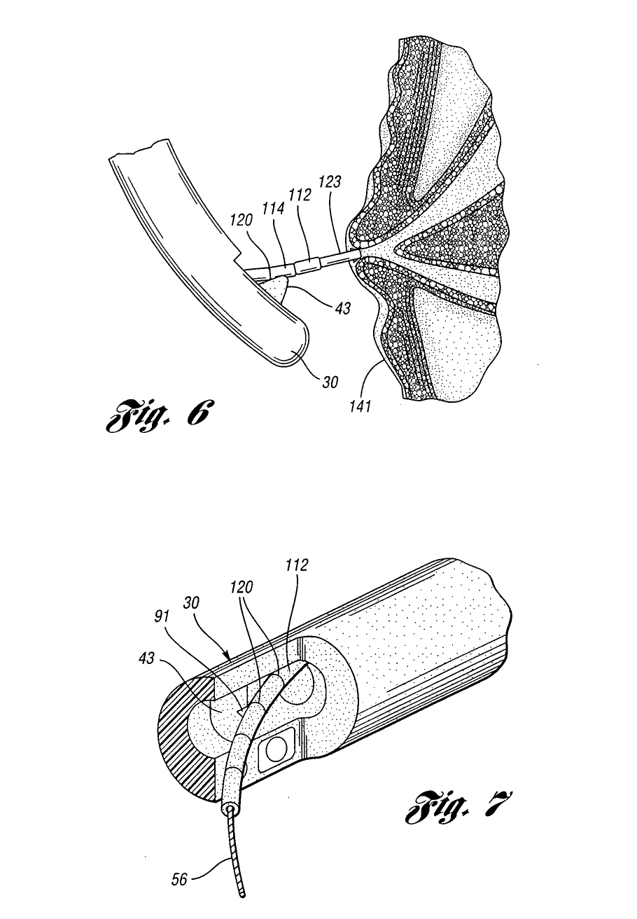

FIGS. 6 and 7 depict the outer catheter 112 in contact and received within the elevator 43 of the endoscope. As shown, the outer catheter 112 is received preferably by the the elevator 43 and is held therein such that movement of other components such as a wire guide is facilitated, thereby deploying a medical device such as the stent 123 to the target location in the patient anatomy 141 or 143. The grooves 120 are formed along the radial axis of the catheter 112 to allow the clinician to longitudinally move a wire guide 56 (or inner catheter) as needed or desired within the patient anatomy. In use, the elevator 43 is preferably actuated to move the elevator 43 away from the distal tip of the endoscope, thereby moving the outer catheter 112. The outer catheter 112 may then be held within the grasping slot as the wire guide 56 or an inner catheter is retracted therethrough to deploy the stent 123. As a result, the grooves 120 allow the outer catheter 112 to be held within the grasping slot 91 to allow for more precise and accurate deployment of the medical device in the patient anatomy.

The embodiments of the present invention discussed herein may be used with the embodiments discussed in the U.S. Non-Provisional Application filed on Mar. 5, 2007 entitled “ENDOSCOPIC ELEVATOR APPARATUS.” Such application claims the benefit of U.S. Provisional Application Ser. No. 60/779,182, filed on Mar. 3, 2006, entitled “ENDOSCOPIC ELEVATOR APPARATUS HAVING A POLYMERIC ELEVATOR WITH A GRASPING SLOT” and U.S. Provisional Application Ser. No. 60/779,181, filed on Mar. 3, 2006, entitled “ENDOSCOPE HAVING AN ELEVATOR WITH A GRASPING COVER,” the entire contents of each are incorporated herein by reference.

While the present invention has been described in terms of preferred embodiments, it will be understood, of course, that the invention is not limited thereto since modifications may be made to those skilled in the art, particularly in light of the foregoing teachings.

1. An apparatus cooperable with a grasping slot of an elevator for an endoscope, the apparatus comprising:
an outer catheter including an inner lumen and an outer surface having a radial groove formed thereabout, the radial groove being cooperable with the grasping slot to inhibit longitudinal movement of the catheter with respect to the elevator.

2. The apparatus of claim 1 wherein the catheter comprises a distal portion and a proximal portion and wherein the radial groove is formed along the distal portion of the catheter.

3. The apparatus of claim 1 wherein the catheter comprises a distal portion and a proximal portion and wherein the radial groove is formed only on the distal portion of the catheter.

4. The apparatus of claim 1 wherein the radial groove is a plurality of radial grooves formed circumferentially about the outer surface of the outer catheter, the radial grooves being spaced apart from each other.

5. The apparatus of claim 1 wherein the radial groove is helically formed about the outer surface of the outer catheter, the outer catheter having a distal portion having a distal end, the radial groove being formed on the distal portion and spaced from the distal end of the outer catheter.

6. The apparatus of claim 1 further comprising an elongate member disposed through the inner lumen of the catheter, the elongate member being a wire guide.

7. The apparatus of claim 1 further comprising an elongate member disposed through the inner lumen of the catheter, the elongate member being an inner catheter.

8. An endoscopic system comprising:
an insertion tube extending to a distal tip and including an elevator movably attached thereto, the elevator having an inner side formed thereon defining a grasping slot;
an outer catheter including an inner lumen and an outer surface having a radial groove formed thereabout, the radial groove being cooperable with the grasping slot to inhibit longitudinal movement of the catheter with respect to the elevator; and
an elongate member disposed through the inner lumen of the catheter.

9. The system of claim 8 wherein the catheter comprises a distal portion and a proximal portion and wherein the radial groove is formed only on the distal portion of the catheter.

10. The system of claim 8 wherein the radial groove is formed having a first predetermined shape and wherein the grasping slot has a second predetermined shape cooperable with the first predetermined shape so that the catheter is fixedly received therein to hold the catheter within the grasping slot of the elevator.

11. The system of claim 8 wherein the radial groove is a plurality of radial grooves formed circumferentially about the outer surface of the outer catheter, the radial grooves being spaced apart from each other.

12. The system of claim 8 wherein the radial groove is helically formed about the outer surface of the outer catheter, the outer catheter having a distal end, the radial groove being spaced from the distal end of the outer catheter.

13. A method of engaging an apparatus cooperable with an elevator of an endoscope, the method comprising:
inserting an outer catheter in a patient anatomy, the outer catheter including an inner lumen and an outer surface having a radial groove formed thereabout, the radial groove being cooperable with the grasping slot;
positioning the outer catheter within a target location in the patient anatomy; and
engaging the radial groove with the elevator to inhibit longitudinal movement of the catheter with respect to the elevator.
